(19) United States
(12) Patent Application Publication
(10) Pub. No.: US 2023/0201386 A1
(43) Pub. Date: Jun. 29, 2023

(54) PRODUCING RADIOPHARMACEUTICAL COLD KITS WITHOUT LYOPHILIZATION

(71) Applicant: The Board of Regents of the University of Oklahoma, Norman, OK (US)

(72) Inventors: Hariprasad Gali, Edmond, OK (US); Gregory Nkepang, Oklahoma City, OK (US); Wendy K. Galbraith, Norman, OK (US)

(21) Appl. No.: 18/081,495

(22) Filed: Dec. 14, 2022

Related U.S. Application Data
(60) Provisional application No. 63/294,212, filed on Dec. 28, 2021.

(51) Int. Cl. A61K 51/12; A61K 51/04

(52) U.S. Cl. CPC A61K 51/1241; A61K 51/0497

(57) ABSTRACT

A method for making a radiopharmaceutical cold kit without lyophilization, comprising (1) providing a labeling ligand, a reducing agent, and a bulking agent, and at least one of an antioxidant and an exchange ligand, wherein each of said labeling ligand, reducing agent, bulking agent, antioxidant and exchange ligand is provided in a dry form; and (2) combining and mixing the labeling ligand, the reducing agent, the bulking agent, and at least one of the antioxidant and the exchange ligand to produce a dry powder mixture, wherein the wherein the dry powder mixture is produced without the use of a lyophilization step. The radiopharmaceutical cold kit comprising the dry powder mixture may be stored, or combined with a radionuclide such as Technetium-99m (^{99m}Tc).

### CROSS-REFERENCE TO RELATED APPLICATIONS

The present application claims the benefit of U.S. Provisional Pat. Application Serial No. 63/294,212, filed Dec. 28, 2021, the disclosure of which is hereby incorporated by reference in its entirety.

### BACKGROUND

Each year, approximately 25 million procedures are carried out with Technetium-99m (^{99m}Tc, Tc-99m) radiopharmaceuticals, and this figure is projected to grow at a rate of about 15% per annum. The availability of short lived ^{99m}Tc (half-life: 6 h) from the ⁹⁹Mo/^{99m}Tc generator, as the product of long lived ⁹⁹Mo (half-life: 67 h), is a major factor behind the universal use of this radioisotope. The parent radionuclide, ⁹⁹Mo, is prepared by the fission of ²³⁵U in a nuclear reactor, with a fission yield of 6%. Owing to its multiple oxidation states, ^{99m}Tc has a versatile chemistry, making it possible to produce a variety of complexes with specific desired characteristics, which is a major advantage of ^{99m}Tc for radiopharmaceutical development.

Conventional industrial production of cold radiopharmaceutical kits involves preparing in an inert atmosphere a solution containing all ingredients, except the ^{99m}Tc radionuclide, that are necessary for preparing the radiopharmaceutical, including the labeling ligand to which ^{99m}Tc is to be complexed, adjusting the pH, sterilizing the solution by filtration, dispensing a desired volume of the sterile solution into sterile vials, lyophilizing (freeze-drying) the solution, filling the vials with an inert gas, and capping the product vials.

In the lyophilization process, the contents are first frozen, the surrounding pressure is reduced and enough heat is added to allow the frozen water in the material to sublimate directly from the solid to the gas phase. There are three stages in the lyophilization process: freezing the material, primary drying and secondary drying. Freezing is generally done using a freeze-dryer with a built-in cooling facility, although occasionally material is frozen outside the freeze-dryer. In this step, it is important to freeze the material at a temperature below its eutectic point (i.e. the temperature at which all the three phases, solid, liquid and gas, coexist) to ensure that sublimation rather than melting occurs in subsequent steps. During the primary drying phase, a vacuum is applied to ensure that the water in the substance sublimates. In this initial drying phase, which is slow, about 98% of the water in the material is sublimated, and pressure and temperature are carefully controlled. A condenser chamber is built into the freeze-drying unit to allow the removed water vapor to resolidify. This condenser prevents water vapor from reaching the vacuum pump, thus ensuring the pump’s performance. Condenser temperatures are typically kept below 50° C. The secondary drying phase aims to sublimate the water molecules that are adsorbed during the freezing process (the mobile water molecules are sublimated in the primary drying phase). In this phase, the temperature is raised even higher than in the primary drying phase to break any physico-chemical interactions that may have formed between the water molecules and the frozen material. Usually, the pressure is also lowered in this stage to encourage sublimation. After the freeze-drying process is complete, the chamber of the freeze-dryer is filled with an inert gas such as nitrogen. The vials are then sealed.

^{99m}Tc radiopharmaceuticals are used in many diagnostic procedures. Cold dimercaptosuccinic acid (DMSA) kits, for example, are used clinically in the scintigraphic evaluation of renal parenchymal disorders. Notably, FDA-approved cold DMSA kits have been manufactured and marketed in the US by GE Healthcare. However, since 2015 GE Healthcare has stopped production of cold DMSA kits due to manufacturing issues. The FDA has thus listed this product under the drug shortage list. Currently, DMSA kits are listed as discontinued in the FDA Orange book.

As is evident from the above, lyophilization is a costly and time-consuming step in the production of radiopharmaceutical cold kits. Moreover, the requirement for water in the process of making the kits increases the chances of microbial contamination of the product. A method for making radiopharmaceutical cold kits, which is faster, less costly, and has less chance for contamination, would be desirable. It is to such an improved process that the present disclosure is directed.

### BRIEF DESCRIPTION OF THE DRAWINGS

FIG. 1 is a photograph showing several cold DMSA kits prepared using the disclosed methods.

FIG. 2 is a photograph showing a vial containing ^{99m}Tc-labeled DMSA.

FIG. 3 is a TLC chromatogram of ^{99m}Tc-labeled DMSA.

FIG. 4 is a TLC chromatogram of sodium pertechnetate (Na^{99m}TcO₄).

FIG. 5 shows an HPLC radiochromatogram of ^{99m}Tc-MAG3.

### DETAILED DESCRIPTION

The present disclosure is directed to a new method for producing radiopharmaceutical cold kit (cold radiopharmaceutical kit) for ^{99m}Tc-labeling or other radionuclides that does not use a lyophilization (freeze-drying) step in the production process. All ingredients remain in a dry powdered form throughout the production process, thereby eliminating the necessity of a lyophilization step in the production of the cold kits. The present method uses trituration of active ingredients with the bulk inactive ingredients in an inert atmosphere, sterilization (by steam, ionizing radiation, or nitrogen dioxide) of the bulk powdered mixture, dispensing a desired quantity of the sterile mixture into sterile vials, filling the vials with an inert gas, and capping the product vials.

Before describing various embodiments of the present disclosure in more detail by way of exemplary descriptions, examples, and results, it is to be understood as noted above that the present disclosure is not limited in application to the details of methods, compositions, and apparatus as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and apparatus of the present disclosure, the following terms, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges (e.g., in units of length such as micrometers or millimeters) include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error. Further, in this detailed description, each numerical value (e.g., temperature, thickness, time, mass, volume, concentration, etc.) should be read once as modified by the term “about” (unless already expressly so modified), and then read again as not so modified unless otherwise indicated in context. Unless otherwise stated, the term “about” or “approximately,” where used herein when referring to a measurable value such as an amount, length, thickness, a temporal duration, and the like, is meant to encompass, for example, variations of ± 20%, or ± 10%, or ± 5%, or ± 1%, or ± 0.1% from the specified value, as such variations are appropriate to perform the disclosed methods and as understood by persons having ordinary skill in the art.

As noted above, any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

As used herein, the term “substantially” means that the subsequently described parameter, event, or circumstance completely occurs, or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement (e.g., thickness).

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “active agent” where used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 50% (w/w) pure, or at least 55% (w/w) pure, or at least 60% (w/w) pure, or at least 65% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein “% (w/w)” is used interchangeably with “wt%”. Where used herein % purity generally refers to the total % of the one or more shikimate analogues in a mixture or extract.

The terms “subject” and “patient” are used interchangeably herein and will be understood to refer to a warm-blooded animal, particularly a mammal. Non-limiting examples of animals within the scope and meaning of this term include dogs, cats, rabbits, rats, mice, guinea pigs, chinchillas, horses, goats, cattle, sheep, zoo animals, Old and New World monkeys, non-human primates, and humans, and any other animal susceptible to a contact dermatitis as described herein.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

Where used herein the term “trituration” refers to grinding or blending one compound into another to dilute the ingredients and/or to add volume for processing and handling.

Where used herein the term “portion” refers to an amount that is less than the whole. The term “minor portion” refers to an amount that is less than 50% of the whole.

Where used herein, the term “geometric dilution” refers to a method of mixing components together. In geometric dilution, when two powders with unequal quantities (weight or volume) are mixed, the powder having the smaller quantity (the first powder) is homogeneously mixed with an equal amount of the powder having the larger weight or volume (the second powder) to make a first dilution. The first dilution is then homogeneously mixed with an equal portion of the second powder (or the remainder of the second powder) to form a second dilution. If the second powder has not been consumed (i.e., completely mixed with the first powder), the second dilution is combined with an equal portion, or the remainder, of the second powder. This process is continued until all of the second powder is consumed.

The cold kits of the present disclosure may be supplied with a set of instructions for use. The set of instructions preferably includes information necessary for proper use of the cold kit, such as mixing instructions and/or dosage and timing of administration of the composition disclosed herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Further, all references to one or more embodiments or examples are to be construed as non-limiting to the claims.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject’s type, size, and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term “ameliorate” means a detectable or measurable improvement in a subject’s condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The active agents of the present disclosure can be combined into formulations or treatments that are synergistic. As used herein the terms “synergism,” “synergistic,” or “synergistic effect” refers to a therapeutic effect or result that is greater than the additive effects of each active agent used individually. Presence or absence of a synergistic effect for a particular combination of treatment substances can be quantified by using the Combination Index (CI) (e.g., Chou, Pharmacol Rev, 2006. 58(3): 621-81), wherein CI values lower than 1 indicate synergy and values greater than 1 imply antagonism. Combinations of the inhibitors and antagonists of the present disclosure can be tested in vitro for synergistic cell growth inhibition using standard cell lines for particular cancers, or in vivo using standard animal cancer models. A synergistic effect of a combination described herein can permit, in some embodiments, the use of lower dosages of one or more of the components of the combination. A synergistic effect can also permit, in some embodiments, less frequent administration of at least one of the administered active agents. Such lower dosages and reduced frequency of administration can reduce the toxicity associated with the administration of at least one of the therapies to a subject without reducing the efficacy of the treatment.

The term “coadministration” refers to administration of two or more active agents, e.g., a cardiac-targeted composition as described herein and another active agent. The timing of coadministration depends in part of the combination and compositions administered and can include administration at the same time, just prior to, or just after the administration of one or more additional therapies Coadministration is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating MI, and co-occurring conditions thereof.

Examples of radionuclides that may be used in the embodiments of the present disclosure include, but are not limited to: ¹¹¹In, ¹¹¹At, ¹⁷⁷Lu, ²¹¹Bi, ²¹²Bi, ²¹³Bi, ²¹¹At, ⁶²Cu, ⁶⁷Cu, ⁹⁰Y, ¹²⁵I, ¹³¹I, ¹³³I, ³²P, ³³P, ⁴⁷Sc, ¹¹¹Ag, ⁶⁷Ga, ¹⁵³Sm, ¹⁶¹Tb, ¹⁵²Dy, ¹⁶⁶Dy, ¹⁶¹Ho, ¹⁶⁶Ho, ¹⁸⁶Re, ¹⁸⁸Re, ¹⁸⁹Re, ²¹¹Pb, ²¹²Pb, ²²³Ra, ²²⁵Ac, ²²⁷Th, ⁷⁷As, ⁸⁹Sr, ⁹⁹Mo, ¹⁰⁵Rh, ¹⁴⁹Pm, ¹⁶⁹Er, ¹⁹⁴Ir, ⁵⁸Co, ^{80m}Br, ^{99m}Tc, ^{103m}Rh, ¹⁰⁹Pt, ¹¹⁹Sb, ^{189m}Os, ¹⁹²Ir, ²¹⁹Rn, ²¹⁵Po, ²²¹Fr, ²⁵⁵Fm, ¹¹C, ¹³N, ¹⁵O, ⁷⁵Br, ¹⁹⁸Au, ¹⁹⁹Au, ²²⁴Ac, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ²²⁷Th, ^{125m}Te, ¹⁶⁵Tm, ¹⁶⁷Tm, ¹⁶⁸Tm, ¹⁹⁷Pt, ¹⁰⁹Pd, ¹⁴²Pr, ¹⁴³Pr, ¹⁶¹Tb, ⁵⁷Co, ⁵⁸Co, ⁵¹Cr, ⁵⁹Fe, ⁷⁵Se, ²⁰¹Tl, ⁷⁶Br, ¹⁶⁹Yb, ¹¹⁰In, ¹⁸F, ⁵²Fe, ⁶⁴Cu, ⁶⁸Ga, ⁸⁶Y, ⁸⁹Zr, ^{94m}Tc, ⁹⁴Tc, ¹²⁰I, ¹²³I, ¹²⁴I, ¹⁵⁴⁻¹⁵⁸Gd, ³²F, ¹¹C, ¹³N, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

In non-limiting embodiments, the process disclosed herein can be used to produce cold radiopharmaceutical kits wherein the ligand intended for labeling by the radionuclide (the “labeling ligand”) is selected from Hydroxymethylenediphosphonic acid (HMDP, HDP), Methylene diphosphonate (MDP), ethane-1-hydroxy-1, 1- diphosphonate (EHDP), pyrophosphate, Ethylenediamine pentaacetic acid (DTPA, Na₅DTPA or Na₃CaDTPA), Mercaptoacetyltriglycine (MAG3, MAG₃), N,N-Ethylene-L,L-dicysteine (EC), L,L-ethyl cysteinate dimer (ECD), Dimercaptosuccinic acid (DMSA), N-(2,6-dimethylphenylcarbamoylmethyl)-iminodiacetate (HIDA), [N-(3-bromo-2,4,6-trimethylphenylcarbamoyl)methyl)-iminodiacetic acid] (mebrofenin, bromo-HIDA, Br-IDA), N-(2,6-diethylacctanilido.) iminodiacetic acid (etifenin, EHIDA), diisopropyl iminodiacetic acid (disofenin, DISIDA), trimethylbromoiminoacetic acid (TBIDA), N-(2,6-diisopropylphenylcarbamoylmethyl) iminodiacetate (iprofenin, PIPIDA), Sodium thiosulphate pentahydrate (sulfur colloid), Stannous chloride dihydrate (tin colloid), human serum albumin (HSA) nanocolloid, HSA colloid, HSA microspheres, human immunoglobulin, macroaggregated albumin (MAA), TRODAT, d,1-Hexamethylpropylene amine oxime (d,1-HMPAO), methoxyisobutylisonitrile) (MIBI), 2-Ethoxy ethyl-3,12-dioxa-6,9-diphosphate tetradecane (tetrofosmin), dextran 3-[(2-aminoethyl)thio]propyl 17-carboxy-10,13,16-tris(carboxymethyl)-8-oxo-4-thia-7,10,13,16- tetraazaheptadec-1-yl 3-[[2-[[1-imino-2-(D-mannopyranosylthio)ethyl]amino]ethyl]thio]propyl ether (tilmanocept), hydrazinonicotimamide tyr3 octreotate (HYNIC-TATE) acetate, hydrazinonicotinyl tyr3 octreotide (HYNIC-TOC), Ubiquicidin (UBI), glucoheptanate, phytate, cysteine, thiouracil, diethyldithiocarbamate, mercaptopyridine, mercaptopyrimidine, thiooxine, acetylacetone, pyridoxal, oxine, tropolone, and tetracycline. In particular but non-limiting embodiments further exemplified below, the process can be used to produce cold DMSA kits, MAG3 kits, and DTPA kits.

In non-limiting embodiments, when the labeling ligand is poorly soluble in aqueous medium (e.g., MAG3) or when the labeling reaction takes a long time, the cold kit composition may include an exchange (“transfer”) ligand such as, but not limited to, sodium tartrate, potassium tartrate, sodium potassium tartrate, sodium citrate, potassium citrate, sodium potassium citrate, sodium gluconate, potassium gluconate, sodium potassium gluconate, tricine, or ethylenediaminetetraacetic acid (EDTA) or its salts, for an intermediate trans-chelation step.

In non-limiting embodiments, the reducing agent of the cold kit composition may be stannous (II) chloride dihydrate, stannous tartrate, stannous citrate, stannous oxalate, stannous pyrophosphate, sodium borohydride, dithionite, or ferrous sulfate, potassium boranocarbonate, silanes, and sodium hydride.

In non-limiting embodiments, the cold kit composition may comprise an antioxidant such as but not limited to ascorbic acid or its salts, p-aminobenzoic acid or its salts, gentisic acid or its salts citric acid or its salts, flavones, flavonoids , phenols, and polyphenols.

In non-limiting embodiments, the cold kit composition may comprise a buffer such as, but not limited to, sodium bicarbonate, potassium bicarbonate, sodium potassium bicarbonate, sodium carbonate, potassium carbonate, sodium potassium carbonate, amino acids (e.g., glycine, cysteine), sodium bicarbonate, potassium bicarbonate, sodium potassium bicarbonate, sodium phosphate, potassium phosphate, sodium potassium phosphate, sodium pyrophosphate, potassium pyrophosphate, sodium potassium pyrophosphate, tetrasodium pyrophosphate, tetrapotassium pyrophosphate, sodium acetate, potassium acetate, sodium potassium acetate, and tricine.

In non-limiting embodiments, the cold kit composition may comprise a bulking agent such as, but not limited to, sugar alcohols (e.g., inositol, mannitol, sorbitol, fucitol), monosaccharides (e.g., glucose, fructose, mannose, trehalose), disaccharides (e.g., sucrose, lactose, maltose), calcium salts (e.g., calcium chloride, calcium lactate, calcium gluconate, calcium citrate).

Alcohols, sugar alcohols, alditols, glycols, polyols, saccharides, and polysaccharides used in the presently disclosed compositions may include, for example, cyclitol, acarviocin, aminocyclitol, bornesitol, ciceritol, conduritol, decahydroxycyclopentane, 5-deoxyinositol, dodecahydroxycyclohexane, ononitol, pinitol, pinpollitol, quebrachitol, theogallin, 3,4,5-tri-O-galloylquinic acid, inositol, inositol pentakisphosphate, cis-inositol, D-chiro-inositol, L-chiro-inositol, epi-inositol, neo-inositol, muco-inositol, neo-inositol, scyllo-inositol, sorbitol, threitol, arabitol, galactitol, iditol, volemitol, sorbitol, fucitol, xylitol, lactitol, erythritol, lactitol, maltitol, phytic acid, quinic acid, propylene glycol, 1,2-propanediol, ethylene glycol, low molecular weight polyethylene glycols (e.g., C2-C10), vegetable glycerine, dipropylene glycol, erythulose, glycerol, panthenol, arabinose, bis-HPPP, cellobiose, mannitol, mannose, glucose, allose, altrose, gulose, idose, lactose, maltose, dextrose, galactose, talose, psicose, fructose, sorbose, tagatose, β-d-ribopyranose, α-d-ribopyranose, β-d-ribofuranose, α-d-ribofuranose, sucrose, xylose, trehalose, cytosine glycol, cyclohexane-1,2-diol, aminomethanol, ethyleneglycol, 1,3-propanediol, 1,4-butanediol, 2,2-dimethyl-1-butanol, ethanol, propanol, butanol, pentanol, hexanol, ethynol, acetylenediol, fenticlor, fucitol, gluconic acid, glucic acid, 2-heptanol, 3-heptanol, 2-hexanol, 3-hexanol, ribitol, ethylhexylglycerin, octoxyglycerin, glucuronic acid, glyceraldehyde, glyceric acid, glycerol 3-phosphate, glycerol monostearate, 2-octanediol, pinacol, racemic acid, tartaric acid, uronic acid, xylosan, 1,2-butanediol, 1,3-butanediol, 1,4-butanediol, 2,3-butanediol, 2-methyl-2,4-pentanediol, neopentyl glycol, maltodextrins, raffinose, stachyose, fructo-oligosaccharides, amylose, amylopectin, starch, glycogen, cellulose, hemicellulose, methyl cellulose, methyl ethyl cellulose, pectins, hydrocolloids, sucralose, isomalt, isomaltooligosaccharide, isomaltulose, maltodextrin, and/or polydextrose, and combinations thereof.

Suitable materials which may be used in the compositions as secondary compounds, carriers, vehicles, or solvents include, without limitation, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, polyethylene glycol, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose.

In non-limiting embodiments, the components of the cold kit may comprise the following quantities:
a labeling ligand in a range of from 10 µg to 25 mg per cold kit,
an exchange ligand in a range of 0.1 mg to 50 mg per cold kit,
a reducing agent in a range of 0.1 mg to 2 mg of Sn(II) per cold kit,
an antioxidant in a range of 0.1 mg to 50 mg per cold kit,
a buffer in a range of 1 mg to 25 mg per cold kit, and
a bulking agent in a range of 5 mg to 100 mg per cold kit.

In non-limiting embodiments, the following sequences of steps for preparing the cold kits may be used:

(a) tituration of ingredients to form a bulk powder, (b) sterilization of the bulk powder, (c) weighing and filling the sterilized bulk powder into sterile glass vials, (d) filling the vials with an inert gas, and (e) capping, crimping, labeling, and packing the filled vials.

(a) tituration of ingredients to form a bulk powder, (b) weighing and filling the bulk powder into glass vials, (c) filling the vials with an inert gas, capping, crimping, labeling the vials, (d) sterilization of the filled vials, and (e) packing the filled sterilized vials.

### EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

Non-limiting examples of processes for making and using cold DMSA, MAG3, and DTPA kits are shown below.

### Example 1. Dimercaptosuccinic Acid (DMSA) Kits (Using Inositol as Bulking Agent)

A batch of the bulk powder enough for 200 cold DMSA kits was prepared by grinding the ingredients (active ingredients and excipients) using a glass mortar and pestle by two methods (Method 1 and Method 2). The order of ingredient addition differentiates the two methods.

### Method 1

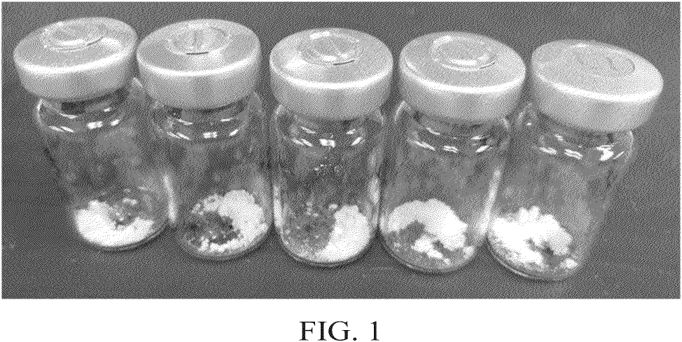

The ingredients were ground starting from the lowest weighed ingredients to the bulkier ingredients using incremental portion addition of the bulkiest weighed ingredient (inositol) to the mixture. Upon addition of each ingredient or portion there was thorough grinding for at least 5 minutes to ensure adequate particle size reduction (to a fine powder) and mixing of the ingredients in the powder mixture. The powder was white in color (see FIG. 1).

The order of addition and their respective masses were as follows:
1) L-Ascorbic acid - 144 mg
2) SnCl₂.2H₂O - 104 mg
3) meso-2,3-Dimercaptosuccinic acid - 203 mg
4) myo-Inositol - 10.06 g (added in 6 successive equal portions)

### Method 2

Same as Method 1, except the order in which ingredient were added during the trituration with the assumption that we do not lose some of the low weighed ingredients through sticking onto the walls of the mortar and pestle. We start with one portion of the bulkiest weighed ingredient (inositol) which is ground for 4 minutes, followed by the lowest weighed ingredients as in Method 1, and finally ending with incremental addition of inositol as the last step. The powder was white in color. The order of addition is as follows:
1) myo-Inositol - 1.3 g
2) L-Ascorbic acid - 141 mg
3) SnCl₂.2H₂O - 104 mg
4) meso-2,3-Dimercaptosuccinic acid - 203.6 mg
5) myo-Inositol - 8.712 g (added successively in 5 equal portions)

After the bulk powder preparation by above two methods, kits were prepared by adding 52 - 53.5 mg of powder to a 5 ml serum vial, purged with nitrogen gas, capped with a butyl rubber septa, and crimped (FIG. 1). Some kits were stored at room temperature and some were stored in the refrigerator at 4° C. to determine the stability of the kits.

Each cold DMSA kit will contain the ingredients as follows:
1) meso-2,3-Dimercaptosuccinic acid - 1 mg
2) SnCl₂.2H₂O - 0.5 mg
3) L-Ascorbic acid - 0.7 mg
4) myo-Inositol - 50 mg

### ^{99m}Tc Labeling

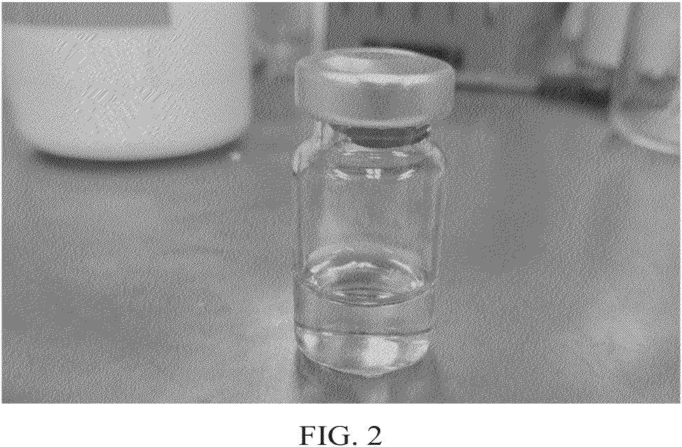

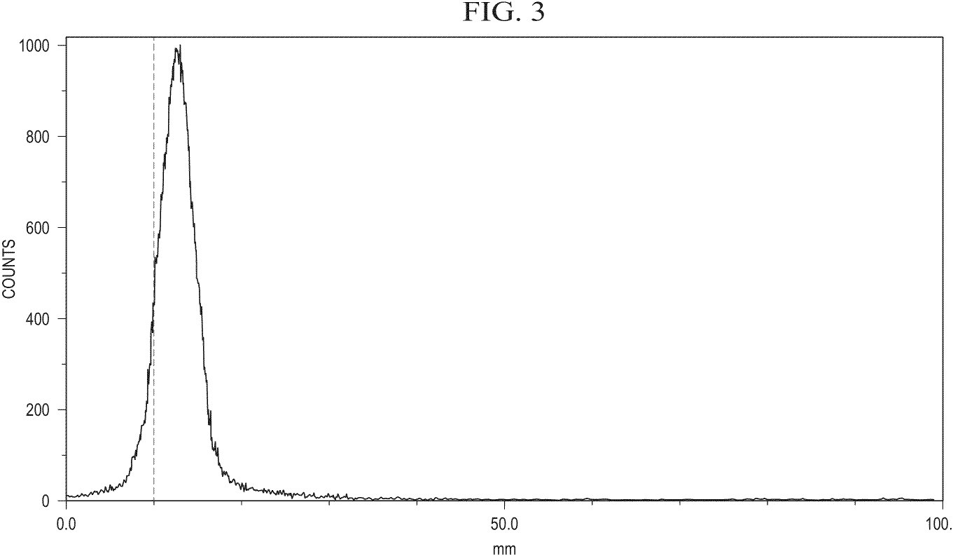

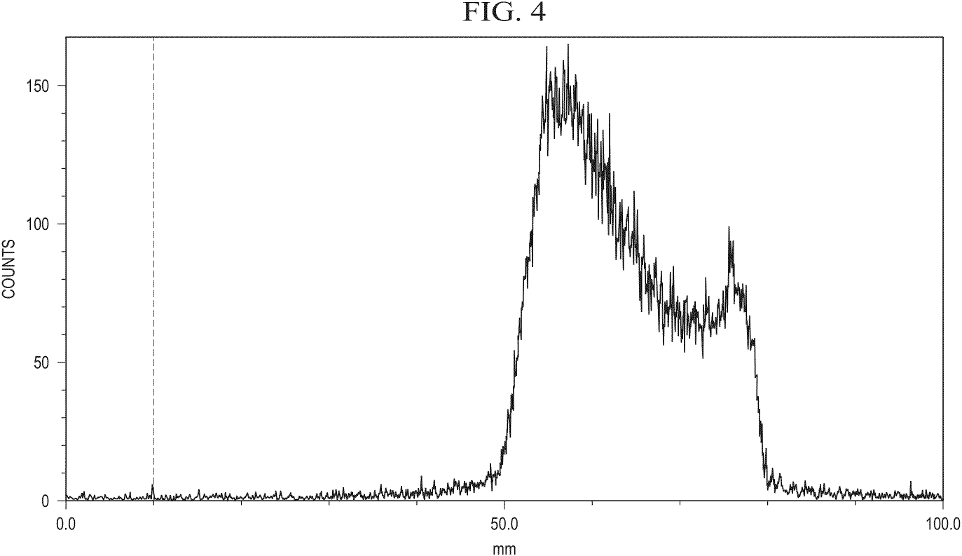

A dose of 25 mCi sodium pertechnetate (Na^{99m}TcO₄) dissolved in 3 ml of normal saline was added to the cold DMSA kit vial. All the powder was dissolved immediately upon addition of the Na^{99m}TcO₄. The solution was incubated for 10 minutes at room temperature. Radiochemical purity (RCP) of the product was determined using thin-layered chromatography (TLC) on a silica gel plate and Acetone as the mobile phase. With these TLC conditions, the product will stay at the bottom and the unreacted pertechnetate (^{99m}TcO₄⁻) will move to the top with the solvent front. Appearance of the product (obtained from kits prepared by both methods) was clear and colorless (FIG. 2), pH was 2.2 - 2.4, and the RCP was >99.9% (FIGS. 3-4).

All the kits (prepared by both methods) were found to be stable at the room temperature as well as at 4° C. for at least the tested period of one month. The bulk powder was also found to be stable (as determined by the radiochemical purity after labeling with ^{99m}Tc) after autoclaving in a sealed vial enclosed in an autoclavable bag for 45 min at 120° C. However, the color of the powder was turned to pale gray after autoclaving.

In conclusion, we found that both methods of bulk powder preparation resulted in the product with the same radiochemical purity. However, Method 2 enables the loss of the lowest weighed ingredients during the trituration to be avoided. This new process enables the production of sterile dry powder cold DMSA kits for clinical use.

### Example 2. DMSA Kits (Using Mannitol as Bulking Agent)

A batch of the bulk powder enough for 200 cold DMSA kits was prepared by grinding the ingredients (active ingredients and excipients) using a glass mortar and pestle by two methods (Method 1 and Method 2). The order of ingredient addition differentiates the two methods.

### Method 1

The ingredients were ground starting from the lowest weighed ingredients to the bulkier ingredients using incremental portion addition of the bulkiest weighed ingredient (mannitol) to the mixture. Upon addition of each ingredient or portion there was thorough grinding for at least 5 minutes to ensure adequate particle size reduction (fine powder) and mixing of the ingredients in the powder mixture. The powder was white in color. The order of addition and their respective masses were as follows:
1) L-Ascorbic acid - 142 mg
2) SnCl₂.2H₂O - 100 mg
3) meso-2,3-Dimercaptosuccinic acid - 202 mg
4) Mannitol - 10.1117 g (added successively in equal 6 portions)

### Method 2

Same as Method 1, except the order in which ingredient were added during the trituration with the assumption that we do not lose some of the low weighed ingredients through sticking onto the walls of the mortar and pestle. We start with one portion of the bulkiest weighed ingredient (mannitol) which is ground for 4 minutes, followed by the lowest weighed ingredients as in Method 1, and finally ending with incremental addition of mannitol as the last step. The powder was white in color. The order of addition is as follows:
1) Mannitol - 1.4 g
2) L-Ascorbic acid - 1141 mg
3) SnCl₂.2H₂O - 104 mg
4) meso-2,3-Dimercaptosuccinic acid - 204 mg
5) Mannitol - 8.712 g (added successively in 5 equal portions)

After the bulk powder preparation by above two methods, kits were prepared by adding 52 - 53.5 mg of powder to a 5 ml serum vial, purged with nitrogen gas, capped with a butyl rubber septa, and crimped. Some kits were stored at room temperature and some were stored in the refrigerator at 4° C. to determine the stability of the kits.

Each cold DMSA kit will contain the ingredients as follows:
1) meso-2,3-Dimercaptosuccinic acid - 1 mg
2) SnCl₂.2H₂O - 0.5 mg
3) L-Ascorbic acid - 0.7 mg
4) Mannitol - 50.6 mg

### ^{99m}Tc Labeling

A dose of 27 mCi Na^{99m}TcO₄ dissolved in 3 ml of normal saline was added to the cold DMSA kit vial. All the powder was dissolved immediately upon addition of the Na^{99m}TcO₄. The solution was incubated for 10 minutes at room temperature. RCP of the product was determined using TLC on a silica gel plate and Acetone as the mobile phase. With this TLC conditions, the product will stay at the bottom and the unreacted sodium pertechnetate will move to the top with the solvent front. Appearance of the product (obtained from kits prepared by both methods) was clear and colorless, pH was 2.2 - 2.4, and the RCP was >99%.

### Example 3. Mercaptoacetyltriglycine (MAG3) Kits

A batch of the bulk powder enough for 200 cold MAG3 kits was prepared by grinding the ingredients (active ingredients and excipients) using a glass mortar and pestle by two methods. The order of ingredient addition differentiated the two methods.

### Method 1

The ingredients were ground starting from the lowest weighed ingredients to the bulkier ingredients using incremental portion addition of the bulkiest weighed ingredient (Potassium Sodium Tartrate) to the mixture. Upon addition of each ingredient or portion there was thorough grinding for at least 5 minutes to ensure adequate particle size reduction (fine powder) and mixing of the ingredients in the powder mixture. The powder was white in color. The order of addition and their respective masses were as follows:
1) SnCl₂.2H₂O - 40.1 mg
2) MAG3 - 200.4 mg
3) Lactose Monohydrate - 4.01 g (added successively in 2 equal portions)
4) Potassium Sodium Tartrate - 10.06 g (added successively in 6 equal portions)

### Method 2

Same as Method 1, except the order in which ingredient were added during the trituration with the assumption that we do not lose some of the low weighed ingredients through sticking onto the walls of the mortar and pestle. We start with one portion of the bulkiest weighed ingredient (Potassium Sodium Tartrate) which is ground for 4 minutes, followed by the lowest weighed ingredients as in Method 1, and finally ending with incremental addition of Lactose Monohydrate and Potassium Sodium Tartrate as the last steps.

The powder was white in color. The order of addition is as follows:
1) Potassium sodium tartrate - 1.2 g
2) SnCl₂.2H₂O - 40.3 mg
3) Mercaptoacetyltriglycine (MAG3) - 200.2 mg
4) Lactose Monohydrate - 4.02 g (added successively in 2 equal portions)
5) Potassium Sodium Tartrate - 8.84 g (added successively in 5 equal portions)

After the bulk powder preparation by above two methods, kits were prepared by adding 71 - 71.3 mg of powder to a 5 ml serum vial, purged with nitrogen gas, capped with a butyl rubber septa, and crimped. Some kits were stored at room temperature and some were stored in a refrigerator at 4° C. to determine the stability of the kits.

Each cold MAG3 kit will contain the ingredients as follows:
1) SnCl₂.2H₂O - 0.2 mg
2) MAG3 - 1 mg
3) Lactose Monohydrate - 20 mg
4) Potassium Sodium Tartrate - 50 mg

### ^{99m}Tc Labeling

A dose of 25 mCi Na^{99m}TcO₄ dissolved in 3 ml of normal saline was added to the cold DMSA kit vial. All the powder was dissolved immediately upon addition of the Na^{99m}TcO₄. The solution was incubated for 10 minutes at 100° C. temperature. RCP of the product was determined using a new two-strip TLC method for routine QC of Mallinckrodt Pharmaceutical ^{99m}Tc-MAG3 labeling based on the first separation on an iTLC-SG chromatography paper with 60:40 Ethyl Acetate:Methyl Ethyl Ketone (MEK) mixture as the mobile phase, combined with the second separation on same support, with a 90:10 Ethanol:Water mixture. With these TLC conditions, the RCP was calculated using the formula:

$$\text{RCP = 100\% - (\% impurities of strip 1 + \% impurities of strip 2)}$$

where
% impurities strip 1 = Top activity X (100/(Top activity + Bottom activity))
Impurities of strip 1: ^{99m}TcO₄⁻ (free pertechnetate) + ^{99m}Tc-MAG2 (lipophilic species of labeled MAG3) both will move to the solvent front, while ^{99m}Tc-MAG3 (product) will remain at the origin.
% impurities strip 2 = Bottom activity X (100/(To activity + Bottom part))
Impurities of strip 2: (^{99m}TcO₂)ₙ (reduced hydrolyzed pertechnetate/colloidal technetium) + ^{99m}Tc-Tartrate (precursor) + ^{99m}Tc-(MAG3)ₓ (precomplexes) will remain at the origin, while free ^{99m}TcO₄⁻ and ^{99m}Tc-MAG3 (product) move to the solvent front using this solvent system.

The appearance of the product was clear and colorless, pH was 5 - 5.5, and the RCP was >90%.

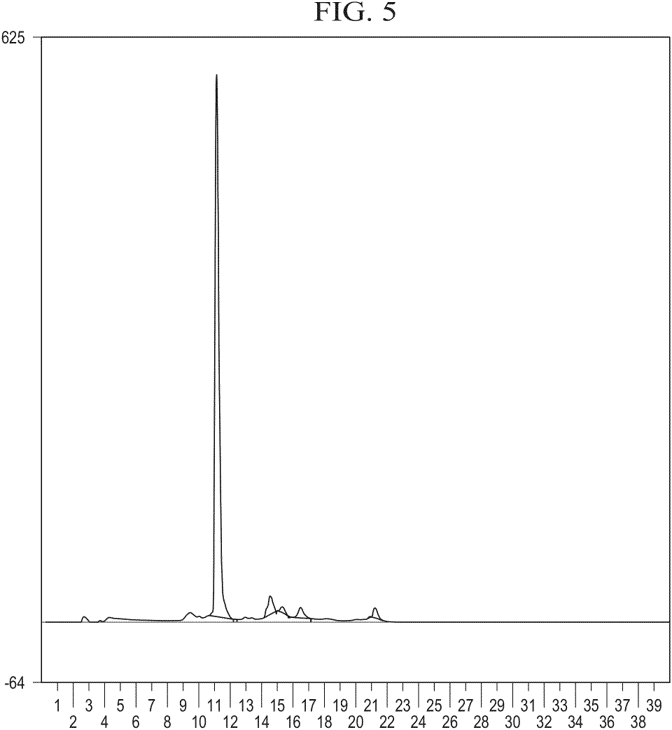

High-performance liquid chromatography (HPLC) was used to quantify the hydrophilic components and hydrophobic components of ^{99m}Tc-MAG3 preparations. Retention time is 11.13 min. HPLC solvents consisted of water containing 0.1% trifluoroacetic acid (solvent A) and acetonitrile containing 0.1% trifluoroacetic acid (solvent B). A Sonoma C18 (ES Industries, 10 µm, 100 Å, 4.6 x 250 mm) column was used with a flow rate of 1.5 mL/min. The HPLC gradient system began with an initial solvent composition of 95% A and 5% B for 2 minutes followed by a linear gradient to 5% A and 95% B in 30 minutes, after which the column was re-equilibrated. FIG. 5 shows a radiochromatogram of the ^{99m}Tc-MAG3 HPLC results. The pure complex appears at retention 11.13 min exhibiting an RCP >90%.

### Example 4. Diethylenetriaminepentaacetic Acid (DTPA) Kits

A batch of the bulk powder enough for 200 cold DTPA kits was prepared by grinding the ingredients (active ingredients and excipients) using a glass mortar and pestle by two methods (Method 1 and Method 2). The order of ingredient addition differentiated the two methods.

### Method 1

The ingredients were ground starting from the lowest weighed ingredients to the bulkier ingredients using incremental portion addition of the bulkiest weighed ingredient (DTPA) to the mixture. Upon addition of each ingredient or portion there was thorough grinding for at least 5 minutes to ensure adequate particle size reduction (fine powder) and mixing of the ingredients in the powder mixture. The powder was white in color. The order of addition and their respective masses were as follows:
1) SnCl₂.2H₂O - 75 mg
2) CaCl₂.2H₂O - 750 mg
3) p-Aminobenzoic Acid - 1 g
4) DTPA - 4.0081 g (added successively in 4 equal portions)

### Method 2

Same as Method 1, except the order in which ingredient were added during the trituration with the assumption that we do not lose some of the low weighed ingredients through sticking onto the walls of the mortar and pestle. We start with one portion of the bulkiest weighed ingredient (DTPA) which is ground for 4 minutes, followed by the lowest weighed ingredients as in Method 1, and finally ending with incremental addition of DTPA as the last step. The powder was white in color. The order of addition is as follows:
1) DTPA - 1 g
2) SnCl₂.2H₂O - 75 mg
3) CaCl₂.2H2O - 752 mg
4) p-Aminobenzoic acid- 1 g
5) DTPA - 3.009 g (added successively in 3 equal portions)

After the bulk powder preparation by above two methods, kits were prepared by adding 29 - 29.2 mg of powder to a 5 ml serum vial, purged with nitrogen gas, capped with a butyl rubber septa, and crimped. Some kits were stored at room temperature and some were stored in the refrigerator at 4° C. to determine the stability of the kits.

Each cold DTPA kit will contain the ingredients as follows:
1) SnCl₂.2H₂O - 0.375
2) CaCl₂.2H₂O - 3.75 mg
3) p-Aminobenzoic acid - 5 mg
4) DTPA - 20 g

### ^{99m}Tc Labeling

A dose of 34.4 mCi Na^{99m}TcO₄ dissolved in 3 ml of normal saline was added to the cold DTPA kit vial. All the powder did not dissolve immediately upon addition of the Na^{99m}TcO₄ but after vigorously shaking the vial upside down for a minute the solid gradually dissolved. The solution was incubated for 15 minutes at room temperature. RCP of the product was determined using a two-strip TLC method for routine QC ^{99m}Tc-DTPA labeling based on the first separation on an iTLC-SG chromatography paper with Saline as the mobile phase, combined with the second separation on the same support, with Acetone. With these TLC conditions, the RCP was calculated using the formula:

$$\text{RCP (\%) = 100\% - (\% impurities of strip 1 + \% impurities of strip 2)}$$

where
% impurities strip 1 (Saline) = Bottom activity X (100/(Top activity + Bottom activity)) Impurities of strip 1: (^{99m}TcO₂)ₙ (reduced hydrolyzed pertechnetate/colloidal technetium) will remain at the origin (Bottom part), while ^{99m}TcO₄ (free pertechnetate) + ^{99m}Tc-DTPA (product) both will move with the solvent front (Top part).
% impurities strip 2 (Acetone) = Top activity X (100/(Top activity + Bottom activity)) Impurities on of strip 2: ^{99m}TcO₄⁻ (free pertechnetate) moves with the solvent front (Top part), while (^{99m}TcO₂)ₙ (reduced hydrolyzed pertechnetate/colloidal technetium) and ^{99m}Tc-DTPA (product) will remain at the origin (Bottom part).

The appearance of the product was clear and colorless, pH was 4.5 - 5.0, and the RCP was >97%.

Long-term stability data for the DMSA, MAG3 and DPTA kits of Examples 1-4 are shown in Table 1. The results demonstrate that there was no loss of labeling efficiency after storage at room temperature (about 22° C.) or cold storage (4° C.) for at least up to 189 days.

TABLE 1

Long-term stability data for DMSA, MAG3 and DPTA kits.

| Kit | Storage Temp | Days² | RCP³ |
|---|---|---|---|
| DMSA (inositol) | RT¹ | 1 | >99 |
| | | 106 | >99 |
| | | 163 | 98.0 |
| | | 189 | 98.5 |
| DMSA | 4° C. | 1 | >99 |
| | | 102 | >99 |
| | | 159 | >99 |
| | | 185 | >99 |
| DMSA (mannitol) | RT | 1 | >99 |
| | | 83 | >99 |
| MAG3 | RT | 1 | 90.3 |
| | | 105 | 91.7 |
| DTPA | RT | 1 | 97.3 |
| | | 91 | 97.6 |

¹=Room temperature; ²= No. of days after production; ³=% Radiochemical purity

### Example 5. Scaled Up Production of the Cold Kit Bulk Powder

In Examples 1-4 the cold kit bulk powder is produced by manually grinding the components in a series of steps. However, the production of the bulk powder can be scaled up to more easily produce larger quantities. In a non-limiting embodiment, the following process can be used.

In the first step, a plurality of ingredients, including for example, a labeling ligand, a reducing agent, and a bulking agent, and at least one of an antioxidant and an exchange ligand, are provided, the particle size of each ingredient is then reduced separately to the same size range, e.g., to a particle size in a range of about 35 µm to 150 µm, to prevent separation of the particles when all the ingredients are mixed together. Thus, each ingredient is first finely ground separately by using a ball mill, hammer mill, colloid mill, roller mill, or other suitable method in a process called comminution (also termed as grinding or pulverization), in which large solid units are reduced into smaller unit mass, coarse particles, or fine particles. Smaller particle size and increased surface area can lead to a uniform distribution of each ingredient in the bulk powder mix. The particle size reduction step is then followed by sieving, weighing each ingredient, and mixing. The powders could be mixed by a special motorized powder blender in which the powder is mixed by tumbling in a rotating chamber rotated by an electric motor. These types of blenders are widely employed in the pharmaceutical industry to perform large volume powder mixings. It is important to place the lighter powder first in the blender and then the heavier one on top of it when mixing powders with different densities. In addition, the method of geometric dilution may be applied when mixing small amounts of an ingredient into a large volume of bulk powder.

### Example 6. Scaled Up Production of the Cold Kit Bulk Powder

In an alternate embodiment, the present disclosure is directed to a process for making a radiopharmaceutical cold kit by providing a plurality of ingredients, including for example, a labeling ligand, a reducing agent, and a bulking agent, and at least one of an antioxidant and an exchange ligand, reducing each ingredient to a fine powder having a particle size of in a range of about 35 µm to 150 µm, sieving each ingredient separately, weighing each ingredient separately, mixing all the weighed powdered ingredients to obtain the final dry bulk powder mix, optionally using geometric dilution for mixing, dispensing a desired quantity of the final dry powder mixture into a sterile vial forming a product vial, filling the product vial with an inert gas, closing the product vial with a sterile stopper, sealing the product vial with an aluminum crimp, and labeling the product vial.

In an alternate embodiment, the present disclosure is directed to a process for making a radiopharmaceutical cold kit by providing a plurality of ingredients, including for example, a labeling ligand, a reducing agent, and a bulking agent, and at least one of an antioxidant and an exchange ligand, reducing the particle size of each ingredient to a fine powder having a particle size of in a range of about 35 µm to 150 µm, sieving each ingredient separately, weighing each ingredient separately, mixing all the weighed powdered ingredients to obtain the dry bulk powder mix, optionally using geometric dilution for mixing, sterilizing the dry bulk powder mix to obtain the final sterile dry bulk powder mix, dispensing a desired quantity of the final sterile dry powder mixture into a sterile vial forming a product vial, filling the product vial with an inert gas, closing the product vial with a sterile stopper, sealing the product vial with an aluminum crimp, and labeling the product vial.

In an alternate embodiment, the present disclosure is directed to a process for making a radiopharmaceutical cold kit by providing a plurality of ingredients, including for example, a labeling ligand, a reducing agent, and a bulking agent, and at least one of an antioxidant and an exchange ligand, reducing the particle size of each ingredient to a fine powder having a particle size of in a range of about 35 µm to 150 µm, sieving each ingredient separately, weighing each ingredient separately, sterilizing each weighed powdered ingredient separately, mixing all the weighed powdered ingredients to obtain the final sterile dry bulk powder mix, optionally using geometric dilution for mixing, dispensing a desired quantity of the final sterile dry powder mixture into a sterile vial forming a product vial, filling the product vial with an inert gas, closing the product vial with a sterile stopper, sealing the product vial with an aluminum crimp, and labeling the product vial.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A method for making a radiopharmaceutical cold kit without lyophilization, comprising:
providing a plurality of ingredients including a labeling ligand, a reducing agent, and a bulking agent, and at least one of an antioxidant and an exchange ligand, wherein each of said labeling ligand, reducing agent, bulking agent, antioxidant and exchange ligand is provided in a dry form; and
combining and mixing the labeling ligand, the reducing agent, the bulking agent, and at least one of the antioxidant and the exchange ligand to produce a final dry powder mixture, wherein the final dry powder mixture is produced without the use of a lyophilization step.

2. The method of claim 1, further comprising sterilizing the final dry powder mixture, dispensing a desired quantity of the sterilized final dry powder mixture into a sterile vial forming a product vial, filling the product vial with an inert gas, and closing the product vial.

3. The method of claim 1, wherein the combining and mixing step comprises:
(1) combining and mixing the labeling ligand and the reducing agent in the dry form to produce a first dry mixture, (2) combining and mixing the first dry mixture and the antioxidant and/or the exchange ligand in the dry form to produce a second dry mixture, and (3) combining and mixing the second dry mixture and the bulking agent in the dry form to produce the final dry powder mixture.

4. The method of claim 1, wherein the order of combining the plurality of ingredients from first to last is (1) the antioxidant, (2) the reducing agent, (3) the labeling ligand, and (4) the bulking agent.

5. The method of claim 1, wherein the order of combining the plurality of ingredients from first to last is (1) a minor portion of the bulking agent, (2) the antioxidant, (3) the reducing agent, (4) the labeling ligand, and (5) the remaining portion of bulking agent.

6. The method of claim 1, wherein the order of combining the plurality of ingredients from first to last is (1) the reducing agent, (2) the labeling ligand, (3) the bulking agent, and (4) the exchange ligand.

7. The method of claim 1, wherein the order of combining the plurality of ingredients from first to last is (1) a minor portion of the exchange ligand, (2) the reducing agent, (3) the labeling ligand, (4) the bulking agent, and (5) the remaining portion of the exchange ligand.

8. The method of claim 1, further comprising providing a buffer, wherein the final dry powder mixture comprises the buffer.

9. The method of claim 8, wherein the order of combining the plurality of ingredients from first to last is (1) the reducing agent, (2) the buffer, (3) the antioxidant, and (4) the labeling ligand.

10. The method of claim 8, wherein the order of combining the plurality of ingredients from first to last is (1) a minor portion of the labeling ligand, (2) the reducing agent, (3) the buffer, (4) the antioxidant, and (5) the remaining portion of the labeling ligand.

11. The method of claim 1, wherein the radiopharmaceutical cold kit comprises an amount of the labeling ligand in a range of about 10 µg to about 25 mg.

12. The method of claim 1, wherein the radiopharmaceutical cold kit comprises an amount of the exchange ligand in a range of about 0.1 mg to about 50 mg.

13. The method of claim 1, wherein the radiopharmaceutical cold kit comprises an amount of the reducing agent in a range of about 0.1 mg to about 2 mg of Sn(II).

14. The method of claim 1, wherein the radiopharmaceutical cold kit comprises an amount of the antioxidant in a range of about 0.1 mg to about 50 mg.

15. The method of claim 1, wherein the radiopharmaceutical cold kit comprises an amount of the bulking agent in a range of about 5 mg to about 100 mg.

16. The method of claim 8, wherein the radiopharmaceutical cold kit comprises an amount of the buffer in a range of about 1 mg to about 25 mg.

17. The method of claim 1, wherein the labeling ligand is selected from the group consisting of Hydroxymethylenediphosphonic acid (HMDP, HDP), Methylene diphosphonate (MDP), ethane-I-hydroxy-I, 1- diphosphonate (EHDP), pyrophosphate, Ethylenediamine pentaacetic acid (DTPA, NA₅DTPA or Na₃CaDTPA), Mercaptoacetyltriglycine (MAG3, MAG₃), N,N-Ethylene-L,L-dicysteine (EC), L,L-ethyl cysteinate dimer (ECD), Dimercaptosuccinic acid (DMSA), N-(2,6-dimethylphenylcarbamoylmethyl)iminodiacetate (HIDA), (3-bromo-2,4,6-trimethylphenylcarbamoyl)methyliminodiacetic acid (mebrofenin, bromo-HIDA, Br-IDA), N-(2,6-diethylacetanilido) iminodiacetic acid (etifenin, EHIDA), diisopropyl iminodiacetic acid (disofenin, DISIDA), trimethylbromoiminoacetic acid (TBIDA), N-(2,6-diisopropylphenylcarbamoylmethyl) iminodiacetate (iprofenin, PIPIDA), Sodium thiosulphate pentahydrate (sulfur colloid), Stannous chloride dihydrate (tin colloid), human serum albumin (HSA) nanocolloid, HSA colloid, HSA microspheres, human immunoglobulin, macroaggregated albumin (MAA), TRODAT, d,1-Hexamethylpropylene amine oxime (d,1-HMPAO), methoxyisobutylisonitrile) (MIBI), 2-Ethoxy ethyl-3,12-dioxa-6,9-diphosphate tetradecane (tetrofosmin), hydrazinonicotimarnide tyr3 octreotate (HYNIC-TATE) acetate, hydrazinonicotinyl tyr3 octreotide (HYNIC-TOC), Ubiquicidin (UBI), glucoheptanate, phytate, cysteine, thiouracil, diethyldithiocarbamate, mercaptopyridine, mercaptopyrimidine, thiooxine, acetylacetone, pyridoxal, oxine, tropolone, and tetracycline.

18. The method of claim 1, wherein the reducing agent is selected from the group consisting of stannous (II) chloride dihydrate, stannous tartrate, stannous citrate, stannous oxalate, stannous pyrophosphate, sodium borohydride, dithionite, and ferrous sulfate.

19. The method of claim 1, wherein the antioxidant is selected from the group consisting of ascorbic acid and its salts, p-aminobenzoic acid and its salts, gentisic acid and its salts, citric acid and its salts, flavones, flavonoids, phenols, and polyphenols.

20. The method of claim 1, wherein the exchange ligand is selected from the group consisting of sodium tartrate, potassium tartrate, sodium potassium tartrate, sodium citrate, potassium citrate, sodium potassium citrate, sodium gluconate, potassium gluconate, sodium potassium gluconate, tricine, or ethylenediaminetetraacetic acid (EDTA) or its salts.

21. The method of claim 1, wherein the bulking agent is selected from the group consisting of sugar alcohols, monosaccharides, disaccharides, and calcium salts.

22. The method of claim 8, wherein the buffer is selected from the group consisting of sodium bicarbonate, potassium bicarbonate, sodium potassium bicarbonate, sodium carbonate, potassium carbonate, sodium potassium carbonate, amino acids (e.g., glycine, cysteine), sodium bicarbonate, potassium bicarbonate, sodium potassium bicarbonate, sodium phosphate, potassium phosphate, sodium potassium phosphate, sodium pyrophosphate, potassium pyrophosphate, sodium potassium pyrophosphate, tetrasodium pyrophosphate, tetrapotassium pyrophosphate, sodium acetate, potassium acetate, sodium potassium acetate, and tricine.

23. The method of claim 1, further comprising the step of adding an amount of a radionuclide to the final dry powder of the radiopharmaceutical cold kit, forming a radionuclide-ligand complex.

24. The method of claim 23, wherein the radionuclide is Technetium-99m (^{99m}Tc) and the radionuclide-ligand complex is a ^{99m}Tc-ligand complex.
